(19) United States
(12) Patent Application Publication
(10) Pub. No.: US 2022/0151325 A1
(43) Pub. Date: May 19, 2022

(54) ARTICLE COMPRISING INSULATIVE AND LIGHTWEIGHT MATERIAL

(71) Applicant: Kelley Peregoy, Lillington, NC (US)

(72) Inventor: Kelley Peregoy, Lillington, NC (US)

(21) Appl. No.: 17/528,042

(22) Filed: Nov. 16, 2021

Related U.S. Application Data
(60) Provisional application No. 63/114,610, filed on Nov. 17, 2020.

(51) Int. Cl. A41D 20/00; A61F 7/02; B32B 5/18; B32B 9/04

(52) U.S. Cl. CPC A41D 20/005; A61F 7/02; A61F 2007/108; B32B 9/046; A61F 2007/0253; B32B 5/18

(57) ABSTRACT

The invention is one or more articles, such as a headband or wristband, that include a material capable of imparting lightweight and thermally insulative characteristics to the article. Particularly, the article comprises one or more layers of aerogel material that impart an insulative characteristic so that it can comfortably be worn in summer months and winter months. In addition, the material is ultra lightweight, so that the article is not bulky and can be comfortably worn for long periods of time to reduce user fatigue. Thus, the article can be worn continuously, even during periods of rest and sleep.

### CROSS-REFERENCE TO RELATED APPLICATIONS

The current application claims priority to U.S. Provisional Patent Application No. 63/114,610, filed Nov. 17, 2021, the entire content of which is hereby incorporated by reference.

### TECHNICAL FIELD

The presently disclosed subject matter relates generally to articles that include a material that provides insulative and ultralightweight characteristics. The disclosed material can be used with items in various industries, such as military, sports, hunting, law enforcement, and the like.

### BACKGROUND

Protective articles are designed to shield a wearer from a variety of environmental hazards, such as gunfire, accidental falls, and the like. For example, different types of military protective gear (e.g., vests, helmets, jackets) are commonly worn to shield the wearer from hot and/or cold temperatures and can provide an added level of protection to the wearer. Similarly, football padding and helmets protect the wearer from contact injuries that commonly occur while playing the sport. However, prior art protective articles typically include an insulative layer that adds bulk and weight, inhibiting freedom of movement to the wearer. As a result, the wearer has increased stress when wearing the garment in situations that require high activity. The bulkiness further accelerates the onset of fatigue. It would therefore be beneficial to provide a protective article that is relatively lightweight yet provides adequate thermal protection.

### SUMMARY

In some embodiments, the presently disclosed subject matter is directed to an article comprising: an exterior layer; an interior layer; and a core layer positioned between the exterior layer and the interior layer, wherein the core layer comprises an aerogel.

In some embodiments, the article includes one or more additional layers.

In some embodiments, the core layer has a thickness of about 0.01-10 inches.

In some embodiments, the core layer comprises about 0.1-100 weight percent aerogel and about 0-99.9 weight percent additives, based on the total weight of the layer.

In some embodiments, the additives are selected from one or more pigments, dyes, UV stabilizers, anti-coloring agents, antioxidants, ion exchange agents, tackifiers, antibacterial agents, matting agents, deodorants, or weathering agents.

In some embodiments, the aerogel has a density of about 1-20 ounces/ft³.

In some embodiments, the aerogel has a density of about 3 ounces/ft³.

In some embodiments, the aerogel has a thermal conductivity of 0.03 W/(m·K) in atmospheric pressure to 0.004 W/(m·K) in modest vacuum.

In some embodiments, the aerogel is a silica aerogel.

In some embodiments, the core layer is removable from the article.

In some embodiments, the aerogel can be added or removed from the core layer on demand.

In some embodiments, the article is selected from a headband or a wristband.

In some embodiments, the article incudes an additional protective layer.

In some embodiments, the article is configured as a golf glove.

In some embodiments, the aerogel is configured as an interior layer of the glove or within an interior compartment.

In some embodiments, the presently disclosed subject matter is directed to a kit defined by an article comprising an exterior layer, an interior layer, and a core layer positioned between the exterior layer and the interior layer; and one or more portions of aerogel material, wherein the article comprises one or more openings through which the aerogel can be selectively added or removed from the interior layer.

In some embodiments, the one or more portions of aerogel material each has a thickness of about 0.01-10 inches.

In some embodiments, the one or more portions of aerogel material have a density of about 1-20 ounces/ft³.

In some embodiments, the one or more portions of aerogel material have a density of about 3 ounces/ft³.

In some embodiments, the one or more portions of aerogel material have a thermal conductivity of 0.03 W/(m·K) in atmospheric pressure to 0.004 W/(m·K) in modest vacuum.

In some embodiments, the presently disclosed subject matter is directed to a headband or wristband comprising an exterior layer; an interior layer; and a core layer positioned between the exterior layer and the interior layer, wherein the core layer comprises an aerogel. In some embodiments, the headband or wristband further includes a pressurized gas cylinder attached to the core layer through tubes to cool the core layer on demand.

In some embodiments, the core layer remains cooled for a period of about 4-6 hours.

### BRIEF DESCRIPTION OF THE DRAWINGS

The previous summary and the following detailed descriptions are to be read in view of the drawings, which illustrate some (but not all) embodiments of the presently disclosed subject matter.

FIG. 1 is a perspective view of an article configured as a vest in accordance with some embodiments of the presently disclosed subject matter.

FIG. 2a is a cross-sectional view of a 3-layer article in accordance with some embodiments of the presently disclosed subject matter.

FIG. 2b is a cross-sectional view of a 1-layer article in accordance with some embodiments of the presently disclosed subject matter.

FIG. 3 is a front plan view of an article configured as a jacket in accordance with some embodiments of the presently disclosed subject matter.

FIG. 4 is a side plan view of an article configured as a helmet in accordance with some embodiments of the presently disclosed subject matter.

FIG. 5 is a cross-sectional view of an article in accordance with some embodiments of the presently disclosed subject matter.

FIG. 6a is a perspective view of an article configured as a golf glove in accordance with some embodiments of the presently disclosed subject matter.

FIG. 6b is a side plan view of an article configured as a golf glove in accordance with some embodiments of the presently disclosed subject matter.

FIG. 7 is a cross-sectional view of an article in accordance with some embodiments of the presently disclosed subject matter.

FIG. 8a is a perspective view of a headband in accordance with some embodiments of the presently disclosed subject matter.

FIG. 8b is a front plan view of a headband in accordance with some embodiments of the presently disclosed subject matter.

FIG. 9a is a perspective view of a wristband in accordance with some embodiments of the presently disclosed subject matter.

FIG. 9b is a perspective view of a wristband in accordance with some embodiments of the presently disclosed subject matter.

FIG. 10a is a front plan view of an article comprising an interior core layer compartment in accordance with some embodiments of the presently disclosed subject matter.

FIG. 10b is a front plan view of the article of FIG. 10b comprising an opening in accordance with some embodiments of the presently disclosed subject matter.

FIG. 11 is a front plan view of an article comprising cooling cylinders connected via tubes to the interior core layer in accordance with some embodiments of the presently disclosed subject matter.

### DETAILED DESCRIPTION

The presently disclosed subject matter is introduced with sufficient details to provide an understanding of one or more particular embodiments of broader inventive subject matters. The descriptions expound upon and exemplify features of those embodiments without limiting the inventive subject matters to the explicitly described embodiments and features. Considerations in view of these descriptions will likely give rise to additional and similar embodiments and features without departing from the scope of the presently disclosed subject matter.

Unless defined otherwise, all technical and scientific terms used herein have the same meaning as commonly understood to one of ordinary skill in the art to which the presently disclosed subject matter pertains. Although any methods, devices, and materials similar or equivalent to those described herein can be used in the practice or testing of the presently disclosed subject matter, representative methods, devices, and materials are now described.

Following long-standing patent law convention, the terms “a”, “an”, and “the” refer to “one or more” when used in the subject specification, including the claims. Thus, for example, reference to “a device” can include a plurality of such devices, and so forth.

Unless otherwise indicated, all numbers expressing quantities of components, conditions, and so forth used in the specification and claims are to be understood as being modified in all instances by the term “about”. Accordingly, unless indicated to the contrary, the numerical parameters set forth in the instant specification and attached claims are approximations that can vary depending upon the desired properties sought to be obtained by the presently disclosed subject matter.

As used herein, the term “about”, when referring to a value or to an amount of mass, weight, time, volume, concentration, and/or percentage can encompass variations of, in some embodiments +/−20%, in some embodiments +/−10%, in some embodiments +/−5%, in some embodiments +/−1%, in some embodiments +/−0.5%, and in some embodiments +/−0.1%, from the specified amount, as such variations are appropriate in the disclosed packages and methods.

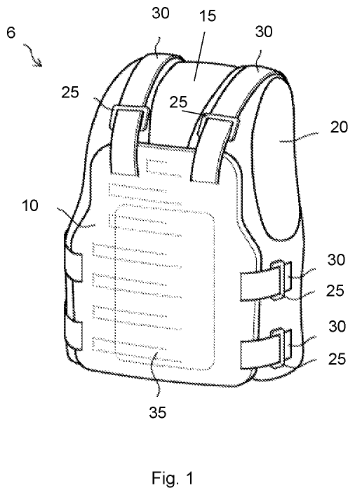

The presently disclosed subject matter is generally directed to one or more articles that include a material capable of imparting lightweight and thermally insulative characteristics to the article. In some embodiments, the article can be a garment worn by a user. For example, FIG. 1 illustrates one embodiment of article 5 configured as a protective vest of the type commonly worn by law enforcement or military personnel. Vest 6 includes front 10 and rear 15 configured with one or more apertures 20 to accommodate the user's arms, torso, and neck. The article can include a variety of optional features such as buckles 25 and straps 30. The article comprises one or more layers of material 35 that impart an insulative characteristic to the vest so that it can comfortably be worn in summer months and winter months. In addition, material 35 is ultra lightweight, so that the article is not bulky and can be comfortably worn for long periods of time to reduce user fatigue. Thus, the article can be worn continuously, even during periods of rest and sleep. The term “ultralightweight” refers to a material with a weight that is substantially less than conventional materials (e.g., conventional materials). The ultralightweight material can have a weight that is 90-99.99 percent less than the conventional material.

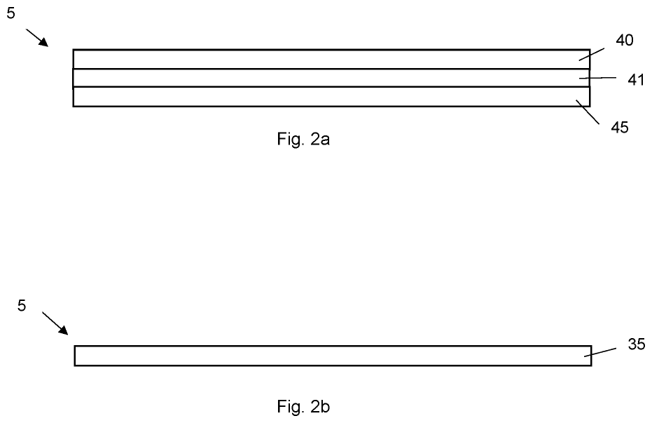

In some embodiments, material 35 can be positioned as core layer 41 of article 5, as shown in FIG. 2a. The term “core layer” refers to a central layer of a multi-layered structure. Any of a wide variety of other materials can be used for inner and outer material layers 40, 45. The term “inner layer” refers to the layer of the material that is in contact with the user's skin or underclothing clothing when worn. The term “outer layer” refers to the outermost layer of the article when worn. The inner and outer layers can include any desired material, such as (but not limited to) cotton, polyester, fleece, linen, denim, rayon, wool, spandex, acrylic, nylon, polymeric material, or blends thereof. In some embodiments, the inner and/or outer layers can be a woven or knit fabric/textile.

In some embodiments, material 35 can be positioned in more than one layer of an article. For example, the presently disclosed subject matter includes embodiments wherein material 35 is configured as inner and/or outer layers 40, 45.

It should be appreciated that the layers of article 5 are not necessarily drawn to scale in the Figures.

Article 5 is not limited to the 3-layer structure shown in FIG. 2a. Rather, the article can comprise any number of layers, such as (but not limited to) 1-100 layers. For example, article 5 can be configured as a single layer, as shown in FIG. 2b.

The thickness of material 35 can vary as desired by the user. For example, a shirt would likely have a thinner layer of material 35 compared to a vest or jacket. Thus, material 35 can have a thickness of about 0.01-10 inches (e.g., at least about (or no more than about) 0.01, 0.05, 0.1, 0.5, 1, 1.5, 2, 2.5, 3, 3.5, 4, 4.5, 5, 5.5, 6, 6.5, 7, 7.5, 8, 8.5, 9, 9.5, or 10 inches. It should be appreciated that the presently disclosed subject matter is not limited and can include articles with material layers thinner or thicker than the ranges set forth above.

In some embodiments, a particular layer of article 5 can comprise about 0.1-100 weight percent of material 35, such as about 25-95 weight percent, 50-85 weight percent, or 60-70 weight percent, based on the total weight of the layer. Thus, an article layer can include at least about (or no more than about) 0.1, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 weight percent material 35, based on the total weight of the layer. The remainder of the layer can include one or more additives and/or can be blended with one or more additional materials (e.g., nylon, cotton, polymeric material, and the like).

The term “additive” refers to any material that is blended or included in a desired layer, such as one or more pigments, dyes, UV stabilizers, anti-coloring agents, antioxidants, ion exchange agents, tackifiers, antibacterial agents, matting agents, deodorants, weathering agents, and the like. Such materials are well known in the art.

Material 35 can include any lightweight material that provides insulative properties. The term “lightweight” refers to the characteristic of being of less weight when compared to corresponding articles constructed from conventional materials. In some embodiments, a lightweight article according to the presently disclosed subject matter has a weight that is at least about 15% lighter than a similar article constructed from standard materials. In some embodiments, material 35 can have a density of about 3 ounces/ft³. Thus, material 35 can have a density of at least about (or no more than about) 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, or 20 ounces/ft³. However, the presently disclosed subject matter also includes embodiments wherein material 35 has a density above or below the ranges given above.

The term “insulative” refers generally to an ability to prevent passage of heat or cold, such as external hot and cold temperatures (e.g., summer and winter weather). Thus, the disclosed material can provide both thermal conduction and convection insulation. Thus, an insulative material has the ability to resist the transfer of heat by conduction, radiation, and/or convection (i.e., to keep the air temperature constant between the skin and article while the outside or environmental air temperature is cooler or warmer). In some embodiments, thermal resistance can be measured by an International Organization for Standardization Test ISO 11092, incorporated by reference herein.

In some embodiments, material 35 comprises an aerogel. The term “aerogel” refers to a synthetic porous ultralight material derived from a gel, where the liquid component for the gel has been replaced with a gas. As a result, aerogels have extremely low density and thermal conductivity characteristics. In some embodiments, aerogel can be produced by extracting the liquid component of a gel through supercritical drying, which allows the liquid to be slowly dried off without causing the solid matrix in the gel to collapse from capillary action. In some embodiments, the aerogel comprises about 99.8 percent air, with a porous solid network that includes air pockets that take up the majority of the space within the aerogel. The lack of solid material allows the aerogel to be approximately weightless.

In some embodiments, the aerogel structure results from a sol-gel polymerization, where monomers react with other monomers to for a sol or a substance comprised of bonded, cross-linked macromolecules with deposits of liquid solution among them. When the material is critically heated, the liquid evaporates and the bonded, cross-linked macromolecular frame is left behind. The result of the polymerization and critical heating is the creation of a material that has a porous strong structure, referred to as an aerogel.

Aerogels nullify conduction and convection, thereby functioning as thermal insulators. Particularly, because aerogels are predominantly constructed from insulating gas, they nullify heat and cool conduction. In addition, the aerogel microstructure prevents net gas movement, nullifying convection. The vast majority of the aerogel structure is made up of one or more gases, which are very poor heat conductors. In addition, air cannot circulation through the aerogel lattice, providing additional convective inhibitors qualities.

Material 35 can comprise any type of aerogel material. For example, material 35 can comprise one or more silica aerogels, derived from silica gel and a modified Stober process. In some embodiments, silica aerogel can have a weight of about 1000 g/m³. The silica solidifies into three-dimensional, intertwined dusters that make up only about 3% of the volume. The remaining 97% of the volume is composed of air in extremely small nanopores. The air has little room to move, inhibiting both convection and gas-phase conduction. Silica aerogel typically has an extremely low thermal conductivity of from about 0.03 W/(m·K) in atmospheric pressure to 0.004 W/(m·K) in modest vacuum, which correspond to R-values of 14 to 105 (US customary) or 3.0 to 22.2 (metric) for 3.5 in (89 mm) thickness.

In some embodiments, material 35 can comprise a carbon aerogel. Carbon aerogels are composed of particles with sizes in the nanometer range, covalently bonded together. They have very high porosity (over 50%, with pore diameter under 100 nm) and surface areas ranging between 400-1,000 m²/g.

In some embodiments, the material can comprise a metal oxide aerogel, such as aerogels made with aluminium oxide.

In some embodiments, the material 35 can comprise one or more Airloy® materials (Aerogel Technologies, Boston, Mass.). Airloys® are ultralight super-insulating materials. Airloys® combine the strength of conventional polymeric materials with the low density and super-insulating characteristics of aerogels. Thus, Airloys® are strong, stiff, and tough and can be used as thermal and acoustic insulators. Airloys® are conventionally 3-10 times lighter when compared to conventional polymeric materials. In some embodiments, a suitable Airloy® has a density of about 0.01-0.9 g/L. Airloys® can be constructed from ceramics, polymers, carbon, metals, carbides, or combinations thereof. Airloys® are generally hydrophobic and thus are stable against moisture and humidity. Airloys® can be optimized for strength-to-weight ratios in excess of 20,000:1 and can vary from strong and rigid to soft and flexible. Airloys® can be electrically insulating with dielectric constants as low as 1.1 with surface areas of 200-800 m² g⁻¹—orders of magnitude higher than conventional materials. Airloys® can also be made electrically conductive with specific surface areas as high as 3000 m² g⁻¹.

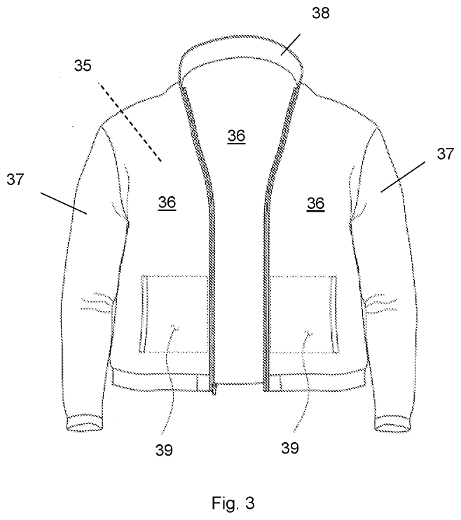

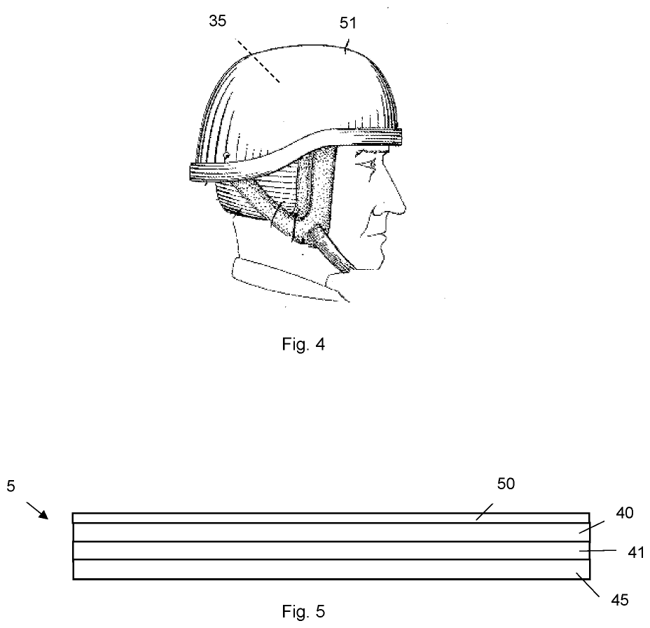

It should be appreciated that the location, dimensions, and design of material 35 within the article can vary as desired to suit a particular application. For example, with the jacket of FIG. 3, it may be desirable to provide material 35 at torso region 36, not in sleeves 37, collar 38, or pockets 39. However, the presently disclosed subject matter also includes embodiments wherein the entire article includes one or more layers of material 35, such as helmet 51 of FIG. 4.

In some embodiments, the layer of material 35 can be adhered, sewn, or otherwise suitably affixed within or to the inner and/or outer layers of article 5. For example, article 5 can be provided with one or more resealable openings to allow for the insertion or deletion of materials 35. In this way, a user can customize an article with regard to weight and/or insulative properties. For example, material 35 can be added around the torso and/or armpits of an article to prevent or reduce the tendency of sweat to accumulate. Further, the number, density, and or size of material layers can be varied to provide different amounts of insulation at different areas as desired by the user. Specifically, the article can include an opening into which material 35 can be inserted and/or removed

Material 35 can be stabilized within article 5 using any known methods, such as through the provision of one or more seams (e.g., non-article forming seams) that secure the material at a desired location. In some embodiments, the greater the number of seams used to secure material 35, the greater the stability of the article and the better the ability of the article to maintain structural integrity when subjected to stresses (e.g., laundering, repeated use). Alternatively, material can be stabilized using any known method, such as mechanical closures (clips, snaps), VELCRO®, ties, and the like.

In some embodiments, layers of material 35 can be stacked and optionally attached together to increase protective and/or thermal regulation capacity. In this way, article 5 can be customized.

The disclosed material 35 is well suited for producing articles that can be used in a variety of fields, such as athletes, military, law enforcement, firefighters, emergency personnel, hunters, outdoor enthusiasts, and the like. For example, article 5 can include (but is not limited to) apparel (e.g., pants, shorts, shirts, sweatshirts, coats, jackets, jerseys, gloves, vests), blankets, sleeping bags, backpacks and other bags, tents, padded items (e.g., knee pads and other protective items), footwear (shoes, boots, socks), and headwear (hats and helmets). As described above, the disclosed articles can be constructed to not only have a lighter weight, but also have excellent insulative qualities while remaining highly breathable.

In some embodiments, article 5 can be reinforced so that it is better able to withstand repetitive wear, use, laundering, and the like. Thus, the disclosed article can include one or more protective layers 50 on at least one surface, as illustrated in FIG. 5. The protective layer can be sewn, releasably attached (e.g., VELCRO®, zipper, snaps), adhesively bonded, and/or heat bonded/fused, to the other layers of the article. In some embodiments, the protective layer can provide waterproof, windproof, or other desirable characteristics.

### ALTERNATE EMBODIMENT

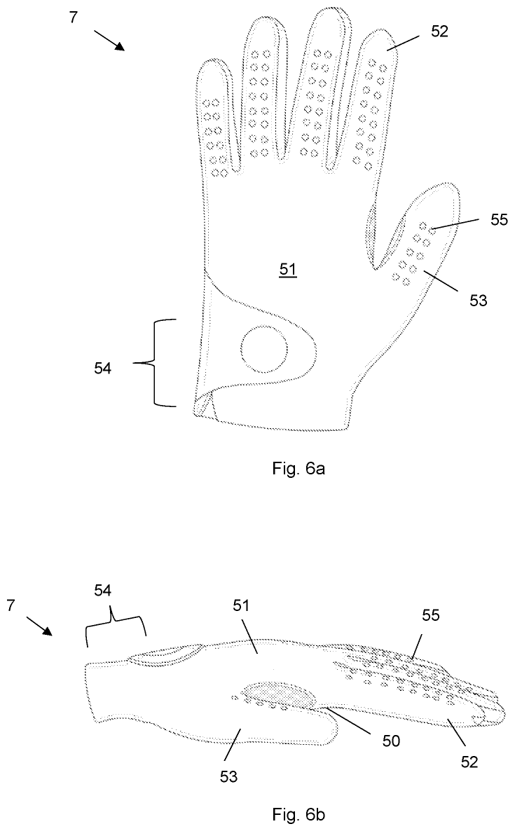

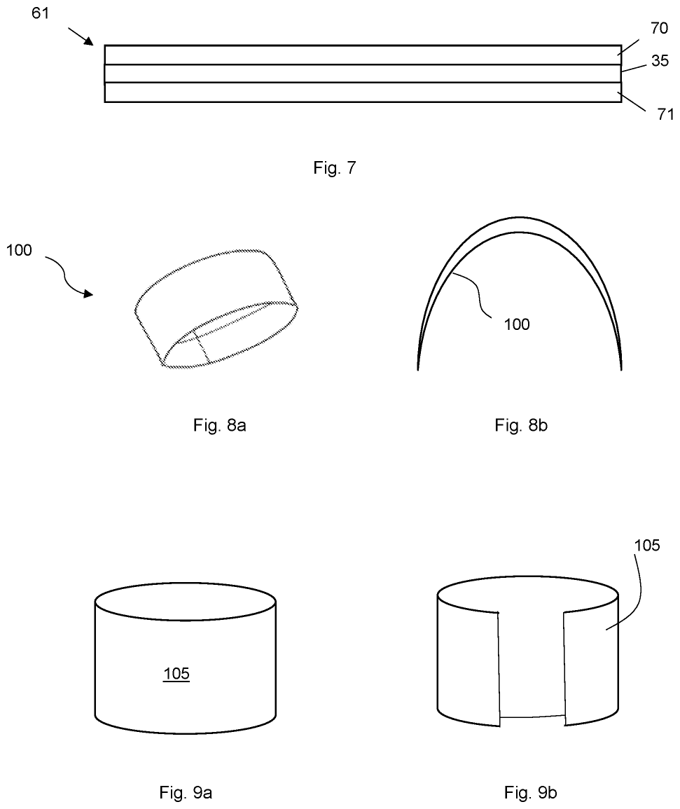

FIGS. 6a and 6b illustrate an alternate embodiment of article 5 configured as golf glove 7. The glove includes palm side 50 and back side 51, a plurality of fingers 52, thumb 53, and wrist portion 54 that can be tightened around a user's wrist. In some embodiments, the glove can include a plurality of ventilation holes 55 positioned on the fingers, thumb, wrist portion, palm side, and/or back side of the glove. In some embodiments, at least one interior layer of glove 7 comprises an aerogel material.

In some embodiments, the glove can be constructed by sewing or attaching top and bottom halves (e.g., palm portion and back side portion) along a perimeter seam. The glove further includes an inside surface that contacts the hand of the user when the glove is worn, and an outside surface that contacts the outside environment (e.g., the golf club).

The glove can be constructed from any known flexible material, such as (but not limited to) leather, synthetic material (e.g., synthetic leather, such as polyurethane coated nylon), fabric, cloth, elastic material, soft plastic, and/or nonwoven material. For example, in some embodiments, Cabretta leather, a well-known material for the manufacture of golf gloves, can be used.

In some embodiments, the glove can have an interior layer that comprises material 35 as described above. For example, as illustrated in Fig. x, the top and/or bottom half of the glove can include an interior layer comprising material 35. As shown, the top half (or bottom half) of the glove further includes contact layer 70 that contacts a surface of the user's hand and external layer 71 that is in contact with the external environment (e.g., the golf club).

In some embodiments, the glove can be configured with an interior compartment comprising material 35. The interior compartment can be configured in one or more locations within the glove interior. For example, in some embodiments, the interior compartment can span the user's palm, fingers, thumb, and/or wrist. The term “compartment” is expansively defined as a structure for storing material 35, and can include a pocket, pouch, receptacle, or combinations thereof. In some embodiments, the compartment contacts the palm-side portion of the user's hand, the back side portion of the user's hand, or both.

Material 35 ensures a proper grip on the golf club when worn and also has an insulating effect on the user's hand during cold or warm weather.

In some embodiments, material 35 can have a thickness of about 1 inch or less. Thus, the material can have a thickness of at least about (or no more than about) 1.0, 0.95, 0.8, 0.85, 0.8, 0.75, 0.7, 0.65, 0.6, 0.55, 0.5, 0.45, 0.4, 0.35, 0.3, 0.25, 0.2, 0.15, 0.1, 0.05, or 0.01 inches.

The present disclosure is not to be limited in terms of the particular embodiments described herein, which are intended as illustrations of various aspects. Many modifications and variations can be made without departing from its spirit and scope, as will be apparent to those skilled in the art. Functionally equivalent methods and apparatuses within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is to be understood that the present disclosure is not limited to particular methods, reagents, compounds, compositions, or biological systems, which can, of course, vary. It is also to be understood that the terminology used herein is for the purpose of describing particular embodiments only and is not intended to be limiting.

### ALTERNATE EMBODIMENT

In some embodiments, the presently disclosed subject matter also includes an article (such as a headband or wristband) worn by a user. The item includes an interior layer comprising one or more gel, liquid, or solid materials. It should be appreciated that the gel, liquid, or solid materials can include any known material or combination of materials.

The term “gel” refers to a composition comprising a viscous polymer having a fluidity at room temperature between that of a liquid and that of a solid. In some embodiments, a gel can include a 3-dimensional porous network obtained by condensation or reaction of one or more precursor particles. Suitable gel materials can include (but are not limited to) hydrogel (network of hydrophilic polymer chains), organogel (non-crystalline, non-glassy thermo-reversible solid material with a liquid organic phase entrapped in a 3-dimensionally crosslinked network), xerogel (solid formed from a gel by drying with unhindered shrinkage), lipogel, hydrophobic gel, hydrophilic gel, nanocomposite gel (hybrid hydrogels, highly hydrated polymeric networks that are physically or covalently crosslinked with each other or nanoparticles or nanostructures, or combinations thereof. Examples of gels that can be used include gelatin, alginate, chitosan, carrageenan, hyaluronate, colloid, silicone, agar, agarose, polyacrylamide, ethylene carbonate, diethylene carbonate, diethyl carbonate, dimethyl carbonate, ethyl-methyl carbonate, diethyl carbonate, and the like.

The term “liquid” refers to a non-gaseous fluid composition, compound, or material that can be readily flowable at the temperature of use (e.g., room temperature). Suitable liquids can include (but are not limited to) water, buffer, salt solutions, glycerin, ethanol, mineral oils, propanol, butanol, pentanol, hexanol, ketone, ester, saline, polyethylene glycol, dimethyl sulfoxide, oils, or combinations thereof.

The term “solid” refers to a non-volatile, non-liquid component, compound, and/or material. Suitable solid materials can include (but are not limited to) sand, pellets, plastic or metal beads, granular metal, mica, wood chips, sawdust, shells, mineral fibers, gravel, or combinations thereof.

The term “headband” refers to any of a variety of clothing accessories worn in the hair and/or around the forehead of a user. Headband 100 can include a loop of elastic material or a horseshoe-shaped piece of flexible plastic or metal, as shown in FIGS. 8a-8b. However, the disclosed headband is not limited and can be configured in an assortment of shapes and sizes.

The term “wristband” refers to any of a wide variety of encircling strips worn on the wrist or lower forearm. Wristband 105 can fully or partially cover the user's wrist, as shown in FIGS. 9a and 9b.

Headband 100 and wristband 105 can be constructed from any desired material, such as (but not limited to) polymeric material, leather, wood, metal, silicone, or combinations thereof. For example, the items can be constructed from a cloth or terrycloth (e.g., absorbent) material. In some embodiments, the headband or wristband can include one or more interior layers as described above for the clothing items.

The headband and/or wristband can have a width that is consistent along a length of the item, as shown in FIG. 8a. Alternatively, the item can taper, as shown in FIG. 8b.

In some embodiments, the gel, liquid, or solid materials can be frozen when the article is placed in a freezer or cooled when the article is placed in a refrigerator. The term “frozen” refers to temperatures below the freezing point of water (e.g., 32 degrees Fahrenheit). The term “cooled” refers to a temperature below that of room temperature (e.g., between freezing and room temperature (between about 32° F. and about 70° F.).

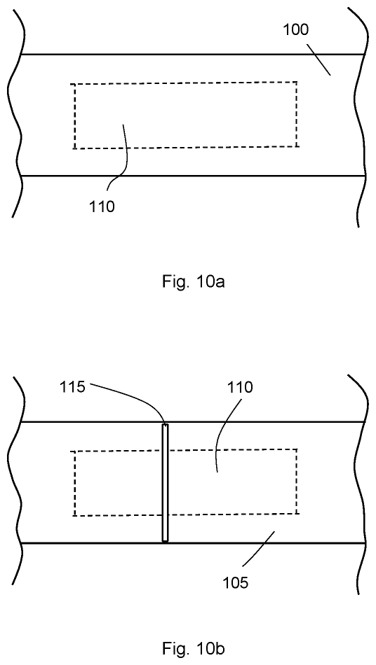

In some embodiments, the gel, liquid, or solid material can be configured within a compartment 110, such as an inner lining, as shown in FIG. 10a. The compartment can be constructed from any known material (e.g., plastic, rubber, thermoplastic material, etc.). The inner compartment can be sewn or otherwise removably or permanently attached to the article. The compartment can optionally include an access 115 to allow a user to contact the interior of the compartment, as shown in FIG. 10b. The access can include any element or elements known in the art, including (but not limited to) buttons, snaps, zippers, VELCRO®, ties, drawstring, and the like.

The remainder of the article can include any material (e.g., fabric) comprising one or more aerogel materials. In some embodiments, the aerogel forms a distinct layer of the article. In other embodiments, the aerogel is blended or infused into the material used to construct the article (e.g., making up about 5, 10, 15, 20, 25, 30, 35, 40, 45, 50, 55, 60, 65, 70, 75, 80, 85, or 95 percent of the article on a weight percent basis). In some embodiments, the aerogel is present in thread, making up about 50-100 weight percent of the thread. Any known type of aerogel or aerogel blend can be used.

Thus, the article includes an inner compartment comprising a gel, liquid, or solid material. The remainder (outside portion) of the article comprises one or more materials, including at least one type of aerogel. The article (e.g., headband, sweatband, hat or other article) can be positioned in a freezer or refrigerator. The interior compartment comprising the gel, solid, or liquid material is then allowed to cool or freeze. The user can then remove the article from the freezer or refrigerator for use. The aerogel material(s) present in the remainder of the article function to maintain the cool temperature of the interior compartment for an extended period of time (e.g., at least 2, 3, 4, or more hours). In this way, a user can wear or hold the article and keep cool, even during very warm conditions (e.g., summer). When the user is done using the article, he can position it back inside the freezer or refrigerator until he wants to use it again.

In some embodiments, the disclosed article can include apparel (jacket, shoe, boot, shoe sole), an accessory (headband, bracelet, sweatband), or any other article (pillow, enclosure, sack, etc.). The article can include a layer that houses a portion of heating or cooling material. For example, the article can include an interior compartment or layer that houses liquid nitrogen, dry ice, ice, water, and/or chemicals (e.g., 2 or more chemicals that can be combined on demand to produce a cooling or heating effect). While not limited, the article can be used for subjects with migraine pain, injuries, sore muscles, and the like. For example, a migraine patient can apply the article to the affected area of the head to achieve cooling or heated relief.

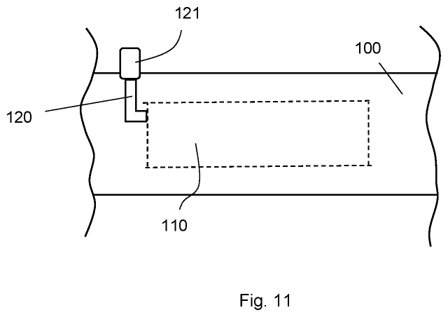

In some embodiments, the article and/or gel can be actively cooled through refrigeration. For example, a small or miniaturized carbon dioxide gas (CO₂) cylinder 121 can be released through tubes 120 to cool the interior of the article (e.g., headband, bracelet, facemask, etc.), as shown in FIG. 11. Any type of cooling element can be used in lieu of carbon dioxide cylinders, such as the use of compressed gases (nitrogen, oxygen, ambient air, helium), liquified gases (LPG, nitrous oxide), dissolved gases (acetylene). In some embodiments, cooling gases such as helium or sulfur hexafluoride can be used. The gas cylinders can be attached to the article such that when released the gas cools the gel to a low temperature. No refrigeration is therefore required. The aerogel provides a layer of protection and insulation such that the article remains cooled for longer periods of time. In addition, the aerogel is lightweight.

In some embodiments, thermal electronics technology can be used, such as use of a Peltier chip. When electricity is added, one side is cooled and the opposed side is heated. Thermoelectric cooling uses the Peltier effect to create a heat flux at the junction of two different types of materials. A Peltier cooler is a solid state active pump that transfers heat or coldness from one side of a device to the other, with consumption of electrical energy, depending on the direction of current. In some embodiments, a voltage is applied across the device, such that the difference in temperature will build up between the two dies.

Solid state refrigeration techniques can also be used, as well as self-contained refrigeration and/or acoustic refrigeration.

It should be appreciated that the disclosed technology can be equally applicable to heat an article, such as during cold conditions.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

The present disclosure may be embodied in other specific forms without departing from its spirit or essential characteristics. The described embodiments are to be considered in all respects only as illustrative and not restrictive. The scope of the present disclosure is, therefore, indicated by the appended claims rather than by the foregoing description. All changes which come within the meaning and range of equivalency of the claims are to be embraced within their scope.

What is claimed is:

1. An article comprising:
an exterior layer;
an interior layer; and
a core layer positioned between the exterior layer and the interior layer;
wherein the core layer comprises an aerogel.

2. The article of claim 1, further comprising one or more additional layers.

3. The article of claim 1, wherein the core layer has a thickness of about 0.01-10 inches.

4. The article of claim 1, wherein the core layer comprises about 0.1-100 weight percent aerogel and about 0-99.9 weight percent additives, based on the total weight of the layer.

5. The article of claim 1, wherein the additives are selected from one or more pigments, dyes, UV stabilizers, anti-coloring agents, antioxidants, ion exchange agents, tackifiers, antibacterial agents, matting agents, deodorants, or weathering agents.

6. The article of claim 1, wherein the aerogel has a density of about 1-20 ounces/ft³.

7. The article of claim 6, wherein the aerogel has a density of about 3 ounces/ft³.

8. The article of claim 1, wherein the aerogel has a thermal conductivity of 0.03 W/(m·K) in atmospheric pressure to 0.004 W/(m·K) in modest vacuum.

9. The article of claim 1, wherein the aerogel is a silica aerogel.

10. The article of claim 1, wherein the core layer is removable from the article.

11. The article of claim 1, wherein the aerogel can be added or removed from the core layer on demand.

12. The article of claim 1, comprising an additional protective layer.

13. The article of claim 14, wherein the aerogel is configured as an interior layer of the glove or within an interior compartment.

14. A kit comprising:
an article comprising an exterior layer, an interior layer, and a core layer positioned between the exterior layer and the interior layer; and
one or more portions of aerogel material;
wherein the article comprises one or more openings through which the aerogel can be selectively added or removed from the interior layer.

15. The kit of claim 16, wherein the one or more portions of aerogel material each has a thickness of about 0.01-10 inches, a density of about 1-20 ounces/ft³, or both.

16. The kit of claim 16, wherein the one or more portions of aerogel material have a density of about 3 ounces/ft³.

17. The kit of claim 16, wherein the one or more portions of aerogel material have a thermal conductivity of 0.03 W/(m·K) in atmospheric pressure to 0.004 W/(m·K) in modest vacuum.

18. The kit of claim 16, wherein the one or more portions of aerogel material comprise a silica aerogel.

19. A headband or wristband, defined by: an exterior layer; an interior layer; and
a core layer positioned between the exterior layer and the interior layer, wherein the core layer comprises an aerogel,
wherein the headband or wristband further includes a pressurized gas cylinder attached to the core layer through tubes to cool the core layer on demand.

20. The headband or wristband of claim 19, wherein the core layer remains cooled for a period of about 4-6 hours.
